(19) United States
(12) Patent Application Publication
(10) Pub. No.: US 2014/0039256 A1
(43) Pub. Date: Feb. 6, 2014

(54) TUBE FOR INSPECTING INTERNAL ORGANS OF A BODY

(71) Applicant: ETView Ltd., Misgav (IL)

(72) Inventor: Oren GAVRIELY, Haifa (IL)

(73) Assignee: ETView Ltd., Misgav (IL)

(21) Appl. No.: 13/902,888

(22) Filed: May 27, 2013

Related U.S. Application Data
(63) Continuation of application No. 13/423,291, filed on Mar. 19, 2012, now abandoned.
Application No. 13/423,291 is a division of application No. 10/529,965, filed on Mar. 31, 2005, now abandoned.
(60) Provisional application No. 60/415,550, filed on Oct. 3, 2002.

(51) Int. Cl. A61B 1/267; A61M 16/04; A61B 1/233; A61B 1/24; A61B 1/015; A61B 1/04

(52) U.S. Cl. CPC A61B 1/267; A61B 1/015; A61B 1/042; A61B 1/233; A61B 1/24; A61M 16/04
USPC 600/109; 600/156; 600/155

(57) ABSTRACT

An inspection tube for use in the medical practice, in which sensors, such as a miniature electronic camera, are incorporated in the distal face of the tube. The sensors receive energy supplied via conduits running along the length of the tube, preferably embedded within the wall. Signals of the sensors are transmitted to the rear of the tube where they are fed into receivers. Sensor cleaning can be affected in some embodiments by conducting cleaning agents through a channel in the wall of the tube. An alarm procedure can be affected by comparing sensed pattern with a reference base pattern and defining a critical deviation from the reference base .

### TECHNICAL FIELD OF THE INVENTION

The present invention relates generally to a medical means of monitoring critically ill and anesthetized patients including monitoring ventilated patients. More specifically, the invention is a device for monitoring patient's organs and cavities.

### BACKGROUND OF THE INVENTION

Insertion of tubes into patient's body organs, cavities and tracts is a common procedure in emergency and critical care medicine. An endotracheal tube may be inserted into the trachea of a patient who is in acute respiratory failure or is undergoing general anesthesia. The endotracheal tube must be placed quickly and accurately and positioned with its tip in the mid portion of the patient's trachea to prevent accidental slipping and to provide proper seal and ventilation of both lungs. Similarly, a naso-gastric tube is commonly inserted through the nose or mouth into the stomach of patients who need artificial feeding or evacuation of the content of the stomach. Another tube that is frequently inserted into a body cavity during emergency treatment is the urinary catheter. This catheter is threaded through the urethra into the urinary bladder. The correct placement of these tubes and catheters throughout their use is critically important.

Many patients who are critically ill or undergoing general anesthesia require artificial ventilation. For over 40 years the most common method of providing artificial ventilation has been by pumping compressed air into the patient's lungs through an endotracheal tube. This tube is inserted through the patient's mouth or nose and passed between the vocal cords into the trachea. Alternatively, a tube may be inserted into the trachea through a tracheotomy surgical incision. For oral intubation the operator usually uses a laryngoscope, which consists of a handle and a blade. The operator inserts the blade into the patient's mouth and advances it until its tip lies in the pharynx beyond the root of the tongue. The handle is then used to manipulate the blade and push the tongue out of the way until the epiglottis and the vocal folds can be seen. The tip of the endotracheal tube can then be aimed and pushed between the vocal folds into the trachea. This method of insertion is used in the majority of intubations, but requires skill, training and experience and is only performed by specialized physicians and licensed paramedics.

An alternative method that is often used when difficult intubation is anticipated is over a fiber optic bronchoscope. First the bronchoscope is connected to a light source to provide the needed illumination of the field facing its tip. The shaft of the bronchoscope is then inserted through the endotracheal tube and moved in as far as possible. The tip of bronchoscope is then inserted into the patient's airway and advanced under visualization through the bronchoscope's eyepiece or a video display in between the vocal folds into the trachea. The endotracheal tube can now be pushed down the bronchoscope shaft and moved between the vocal folds into the trachea. The endotracheal tube can now be secured and the bronchoscope removed to free up the lumen of the endotracheal tube. While the bronchoscopic method is safer than with the laryngoscope, the equipment needed is expensive, delicate and more cumbersome and is seldom found in the field or on emergency medical vehicles.

Securing the endotracheal tube and preventing its inadvertent movement during use is critical to the prevention of dire accidents. Inflating a cuff that surrounds the tube near its tip occludes the space between the outer wall of the tube and the inner wall of the trachea to provide an airtight seal. The cuff is connected to the external end of the endotracheal tube through a thin channel to in the tube's wall. The channel is connected to a one-way valve through which air can be injected to inflate the cuff to the desired pressure and volume. The cuff is also helpful in securing the tube in place, but additional fasteners are usually applied around the head to prevent the tube from slipping in or dislodging.

Once the tube has been inserted, it is mandatory to verify its correct position. Accidental insertion of the tube into the esophagus or placing it too deep inside the airways, so that its tip is lodged in one of the main stem bronchi instead of in the trachea may lead to catastrophic consequences and asphyxiation. Many methods are available to verify the endotracheal tube placement. Auscultation of both sides of the chest is usually done to verify symmetric air entry into both lungs. A chest x-ray is another well-tested method of verifying the tube placement. The x-ray picture reveals the relationships between the endotracheal tube tip and the tracheal first bifurcation (carina). X-ray pictures may be and should be taken whenever an endotracheal tube is placed or repositioned. Additionally, the tube placement may be verified through a fiber optic bronchoscope, by a suction bulb, or through sending and receiving an acoustic signal. These methods are used to verify the initial placement of the endotracheal tube. There are no currently available means for continuous monitoring of the actual placement of the tube.

The advantages of fiber optic visualization were combined with the simple design of the laryngoscope as disclosed by several patents and scientific papers. Additionally, the use of visualization stylets which include means for seeing the airways during the insertion of an endotracheal tube have been described. However, there are no known methods for incorporating the visualization means permanently into the anterior face of the endotracheal tube so that visualization of the airways can be accomplished during the insertion and continuously thereafter.

### BRIEF DESCRIPTION OF THE DRAWINGS

FIG. 1 is a schematic isometric scheme of the tube of the invention incorporating three types of conduits;

FIG. 2 is a schematic isometric description of a portion of the anterior face of the tube of the invention into which a miniature video camera is incorporated;

FIG. 3 is a schematic description of the items commuting along the tube of the invention, related to the performance of inspection tasks.

DESCRIPTION OF THE PRESENT INVENTION

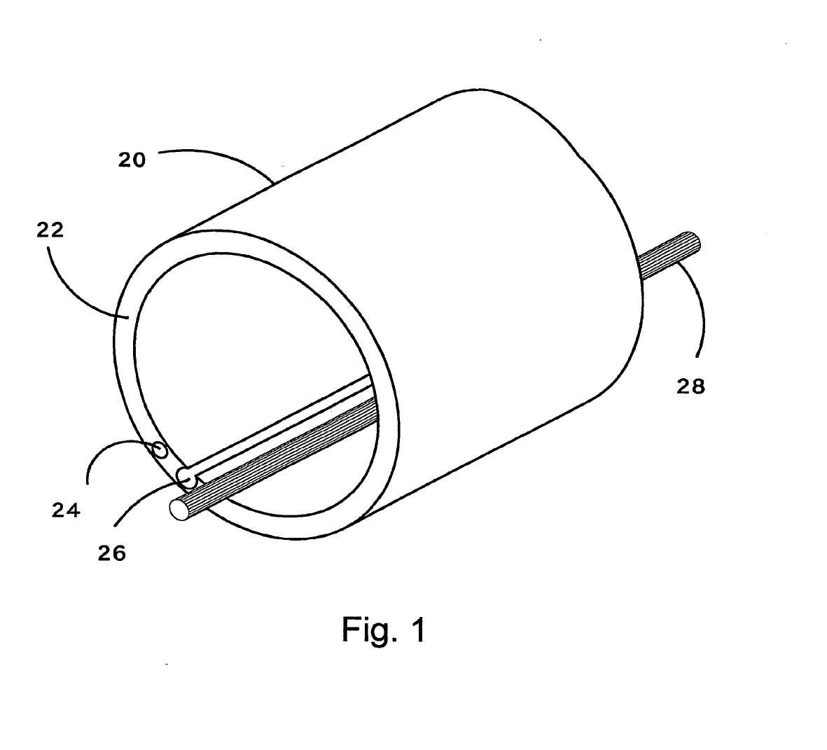

In accordance with the present invention, a multifunctional inspection tube is provided for collecting information about internal cavities and spaces in the body of a patient or an animal in association with the insertion of an inspection tube in the body. The multifunctional inspection tube is a modified medical tube such as an endotracheal tube, catheter, a gastric feeding tube. In accordance with the present invention the tube is equipped with means to examine both the positioning of the inspection tube with respect to body organs and the functional aspects of the body during and after the insertion. Thus, the tube of the invention may be used to perform not only customary medical treatment tasks of conveying gasses and or liquids to and from the penetrated organs, but also inspection tasks that examine the reaction to such treatment and otherwise the condition of the penetrated organs. The multifunctional inspection tube of the invention incorporates a means of receiving signals relating to the condition of the penetrated organs such as visual and audio signals by employing suitable sensors incorporated at or near the anterior face of a tube. The signals produced by the sensors are transmitted via wires or communication fibers running along the length of the tube to a connector or a wireless transmitter located at the posterior portion of the tube near its standard connector to the ventilation source, gastric tube feeder or urinary collecting device. The signals are received by a receiver containing a suitable signal conditioning means for subsequent processing, display, recording and or monitoring. The structural concept of the invention is better explained with reference to FIG. 1. A portion 20 of a multifunctional inspection tube of the invention is shown, including an anterior face 22. Channels and a conductor are associated with the wall of the tube. A totally embedded channel 24 runs along the length of the inspection tube within its wall, alongside an open recessed channel 26. A conducting element 28 runs along the length of the tube without being embedded in the wall of the tube, rather it is attached to the wall of the tube and occupies a space in the lumen of the tube. The conduit may be partially embedded in the tube or it may be inserted within a recess or it may be threaded within a totally embedded channel without being attached to the tube. Even in embodiments in which the lumen contains a conducting element attached to the wall as described above, the lumen of the tube is still largely free for transferring liquids or gasses in both directions. With this respect, embodiment in which the channels and conducting elements embedded or wholly inserted in the wall may be preferable.

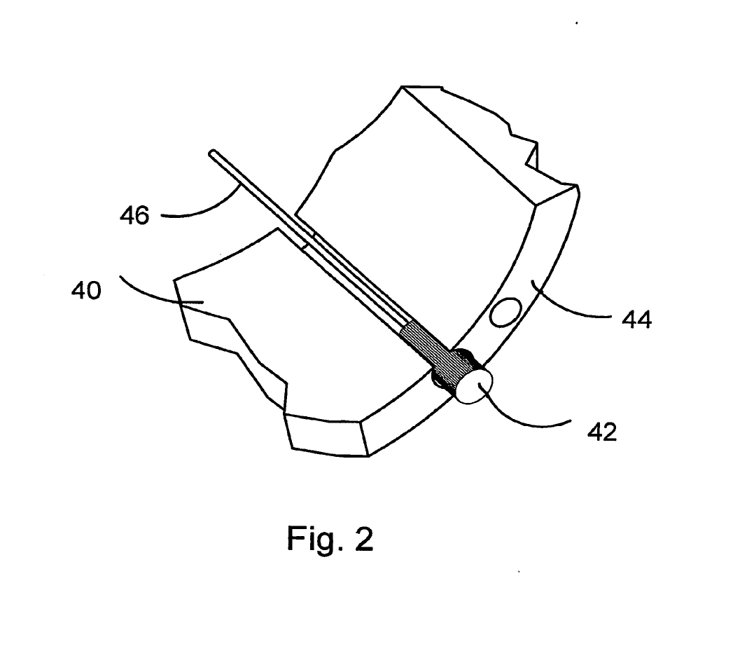

A typical feature of the multifunctional inspection tube is the acquisition of internal images of the body. For acquiring the images, an image sensor may be employed, such as a miniature electronic camera employing a CCD or a CMOS chip incorporated in the anterior face of the inspection tube. In one embodiment, the camera is incorporated in a recess in the wall of the tube as described in FIG. 2 to which reference is now made. A portion of an inspection tube is shown, within the inner side 40 of which, a camera 42 is inserted in a recessed channel, protruding from the anterior face 44 of the tube. The signal of the camera is transmitted by a conducting element 46, typically a copper wire or an optical fiber. The camera's lens is facing away from the tube. The signals arriving from the camera are subsequently fed to a receiver and may be subsequently displayed on a screen, which may be a stand-alone mini screen, an ordinary video screen, or a portion of the display screen ordinarily used to monitor the physiological parameters and well-being of the patient. In some embodiments of the invention, a fiber optical element running along the length of the tube is used to convey light to illuminate the field of view ahead of the anterior face of the inspection tube. Alternatively, a light source may be associated with the proximal face of the tube. Examples for light sources are miniature halogen lamps, light emitting diodes (LED), lasers, or any other kind of light-emitting source of suitable size. An alternative method of illumination is by constructing the inspection tube made of light-conducting material. In some embodiments of the invention, means for keeping the lens of the camera and/or other sensors clean and clear are employed. The airways, stomach or urinary bladder of an ill patient are often filled with secretions that may be thick and viscous. Thus, it is quite possible that the secretions may lodge on the lens and obscure its field of view, or on other sensors thereby modifying their responsiveness. To overcome such an obstacle, a constant or intermittent flow of air or physiological fluid is pumped through a channel in the tube's wall, whereby the outlet of the channel is aimed directly over and around the lens or the sensor's active surface. This flow may be generated by a simple flow source or by a device that is triggered to emit flow upon command from a human care giver, a timer or a software program that monitors the signal and determines when clearing action is required.

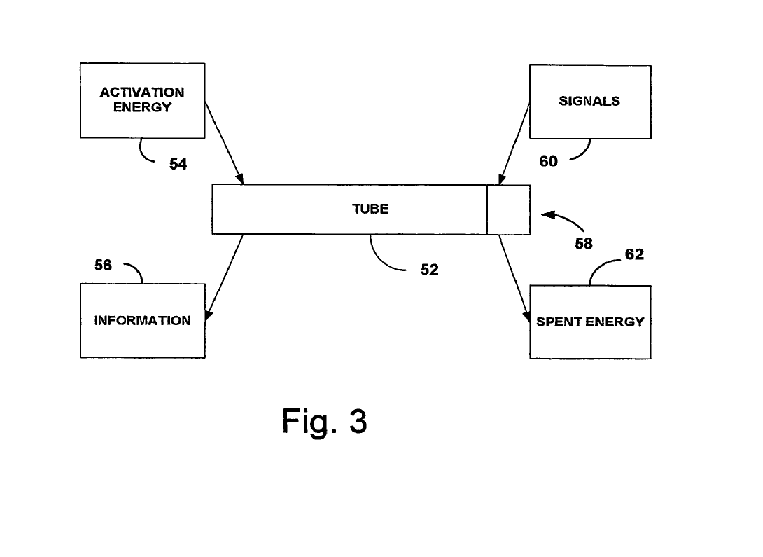

In general, the inspection tube is used as bi-directional conveying platform for various elements required for the fulfillment of its inspection tasks. This is described schematically in FIG. 3 to which reference is now made. Tube 52 receives activation energy 54 of one or several types on its rear end, and downloads information 56, raw or processed at the same end. At the anterior end 58, the tube receives signals 60 of one or several types, and spends energy 62 as will be elaborated later on.

In some embodiments of the invention, a microphone is employed in the tube. Such a microphone can be incorporated in the wall of the tube. Such a microphone receives acoustic signals from at least the vicinity of the tubes anterior, and transfers the signals, raw or processed to the rear of the tube for further downloading and processing.

A plurality of sensors can be effectively employed in the anterior face of the tube of the invention, the non exhaustive list includes cameras, video cameras, microphones, pressure transducers and thermal sensors. Gas sensors, for example sensors for particular gasses such as oxygen and carbon dioxide may also be employed. The energy required to activate such sensors is supplied by conduits of energy such as electric wires incorporated in the tube. In addition, auxiliary energy can be supplied to the vicinity of the anterior face of the tube for the purpose of cleaning and clearing the sensors active facets by flushing them with cleaning media such as gases, humidified air or oxygen, or liquids, typically a physiological solution, through channels in the wall of the tube. Liquids and or gases for flushing are energized and conducted typically via a totally embedded channel. The inspection tube of the invention may be used alone or in combination with other catheters and tubes that are ordinarily inserted into a body organ, tract or cavity such as the esophagus, the stomach, the intestine, the colon, the urinary bladder, the pleural space, lung airways and/or the peritoneal cavity. The present technology may be applied in various medical practices and treatments such as: artificial ventilation of the lung, feeding or removing the content of the stomach, draining urine from the bladder, draining the gas and feces from the colon, and draining or injecting into a surgically accessed cavity such as the pleural space, or the peritoneal cavity.

The sensors of the tube transmit one or more signal types, which are either preprocessed in the sensor for example on the CCD chip, or may be sent raw, to be further processed by analog or digital circuits to yield information relating to the status of the organ or body cavity inspected. The receiving and or processing devices such as, monitors, displays, storage means, analyzers, DSP processors, computers and generators of alarm signals are typically connected by one or a plurality of connectors to the tube. The tube of the invention may be used for insertion through orifices such as the nose, mouth, urethral meatus, rectum, or a surgical incision.

The transmission of raw or preprocessed signals is affected through conductors along the tube such as wires or optical fibers, which connect to a connector at the rear of the tube. A wireless transmitter or transceiver may be applied anywhere suitable on the tube, typically at the rear, for communicating with a console containing a receiver and processor and or a control module.

The inspection tube of the invention may also be used to detect changes in indications of vital functions of a patient. Accordingly, image and acoustic signal are being detected, processed and compared to a reference base picture or sound structure. An alarm is set as soon as certain changes in the indication pass a predetermined threshold. For example, the accumulation of secretions, or development of excessive or diminished lung noises are abnormal.

1. An artificial ventilation system adapted for ventilating a subject, the system comprising a multifunctional endotracheal tube comprising an elongate cylindrical structure having a wall and defining a free lumen for transferring liquids or gasses in two directions, said free lumen being adapted to permanently maintain patency and to maintain ventilation of the subject, wherein said endotracheal tube has a proximal end adapted to protrude from a mouth of the subject and an anterior face;
wherein said tube comprises a conduit located between an inner diameter and an outer diameter of said tube from said proximal end of said tube to said anterior face of said tube, the tube including:
a. an imaging sensor incorporated in said anterior face ;
b. at least one conducting element for transmitting image signals of said imaging sensor to the proximal end of said tube via said conduit, wherein said imaging sensor is continuously acquiring signals and transmitting signals via said conduit; and
c. an image signal receiver for receiving said image signals.

2. The system of claim 1, further comprising a lighting element provided about said anterior face of said tube.

3. The system of claim 2 wherein said lighting element is selected from the group consisting of an optical fiber, halogen lamps, light emitting diodes (LED), lasers, and any combination thereof.

4. The system of claim 3, wherein said lighting element is an optical fiber running along the wall of said tube.

5. The system of claim 1, wherein said imaging sensor is fixedly attached to said conduit.

6. The system of claim 1, wherein said conduit is embedded in said wall.

7. The system of claim 6, further comprising a lighting element located within said conduit.

8. The system of claim 1, wherein said wall comprises a recess and wherein said conduit is located in said recess.

9. The system of claim 1 further comprising cleaning means for keeping said imaging sensor clear.

10. The system of claim 9 wherein said cleaning means are provided through a totally embedded channel.

11. The system of claim 10, wherein said cleaning means are triggered by at least one of a software or a timer.

12. The system of claim 1 further comprising a microphone for receiving acoustic signals from the vicinity of the anterior face of the tube.

13. The system of claim 1 wherein said image sensor is provided in the form selected from the group consisting of: a miniature electronic camera, a CCD and a CMOS.

14. The system of claim 1 wherein said image sensor protrudes from said anterior face of said tube.

15. The system of claim 1 wherein at least a portion of said tube comprises light-conducting materials.

16. The system of claim 1 further comprising suitable sensors incorporated at or near the anterior face of said tube, selected from the group consisting of pressure transducers, gas sensors, oxygen sensor, carbon dioxide sensor an any combination thereof.

17. The system of claim 1 wherein said image sensor is adapted to send signals to display and monitoring devices so as to yield information relating to the status of the respiratory system or organ being penetrated by said tube.

18. The system of claim 17 wherein said image sensor provides data in pre-processed (raw) form.

19. The system of claim 1 adapted for insertion through orifices selected from the group consisting of the nose, mouth or a surgical incision.

20. The system of claim 1, further comprising at least one circuit for processing signals provided by said sensor so as to yield information relating to the status of said biological system or organ penetrated by said tube.

21. The system of claim 20, further comprising means for detecting changes in indication of vital functions comprising means for processing and comparing the image signals to a reference base picture, an alarm to be set as soon as certain changes in the indication pass a predetermined threshold.

22. The system of claim 21 wherein said at least one circuit communicates with said sensor through wireless communication, further comprising a wireless transmitter located at the posterior portion of said tube.

23. The system of claim 21, wherein said at least one circuit communicates with said sensor through wired communication, further comprising a wire connecting said sensor to said at least one circuit.

24. The system of claim 21, further comprising a console containing a receiver and processor and or a control module, said console comprising said at least one circuit.

25. The system of claim 21, further comprising one or more devices selected from the group consisting: monitors, displays, storage means, analyzers, DSP processors, computers, and alarm generators, wherein said one or more devices are in communication with said means for detecting changes.

26. An artificial ventilation system adapted for ventilating a subject, the system comprising a multifunctional endotracheal tube comprising an elongate cylindrical structure having a wall and defining a free lumen for transferring liquids or gasses in two directions, said free lumen being adapted to permanently maintain patency and to maintain ventilation of the subject, wherein said endotracheal tube has a proximal end adapted to protrude from a mouth of the subject and an anterior face and wherein said endotracheal tube is adapted to maintain respiration of said subject;
wherein said tube comprises a conduit located between an inner diameter and an outer diameter of said tube from said proximal end of said tube to said anterior face of said tube, the tube including:
a. means for imaging incorporated in said anterior face ;
b. means for transmitting image signals of said imaging sensor to the proximal end of said tube via said conduit, wherein said means for imaging is continuously acquiring signals and transmitting signals via said conduit, said conduit being adapted to permanently maintain patency;
c. means for receiving said image signals; and
d. at least one connector disposed about the proximal end of said tube for connecting to said means for receiving image signals.

27. A multifunctional endotracheal tube comprising an elongate cylindrical structure having a wall and defining a free lumen for transferring liquids or gasses in two directions, wherein said endotracheal tube has a proximal end adapted to protrude from a body of a patient and an anterior face and wherein said endotracheal tube is adapted to maintain respiration of said patient;
wherein said tube comprises a conduit located between an inner diameter and an outer diameter of said tube from said proximal end of said tube to said anterior face of said tube, the tube including:
a. an imaging sensor incorporated in said anterior face, wherein said imaging sensor is fixedly attached to said conduit ;
b. at least one conducting element for transmitting image signals of said imaging sensor to the proximal end of said tube via said conduit, wherein said imaging sensor is continuously acquiring signals and transmitting signals via said conduit, said conduit being adapted to permanently maintain patency;
c. an image signal receiver for receiving said image signals; and
d. at least one connector disposed about the proximal end of said tube for connecting to said image signal receiver.
